(19) United States
(12) Patent Application Publication
(10) Pub. No.: US 2015/0126545 A1
(43) Pub. Date: May 7, 2015

(54) COMPOSITIONS CONTAINING KINASE INHIBITORS

(71) Applicant: AbbVie Inc., North Chicago, IL (US)

(72) Inventors: Yi Shi, Libertyville, IL (US); Josh M. Lipari, Racine, WI (US); Brian E. Padden, Grayslake, IL (US); Lloyd S. Dias, Gurnee, IL (US); Julie K. Spence, Vernon Hills, IL (US)

(73) Assignee: AbbVie Inc., North Chicago, IL (US)

(21) Appl. No.: 13/704,872

(22) PCT Filed: Dec. 14, 2012
(86) PCT No.: PCT/US12/69641

§ 371 Date: Dec. 17, 2012

Related U.S. Application Data
(60) Provisional application No. 61/570,679, filed on Dec. 14, 2011.

(51) Int. Cl. A61K 31/4365; A61K 9/00; A61K 47/02; A61K 47/28; A61K 47/10; A61K 47/44

(52) U.S. Cl. CPC A61K 31/4365; A61K 47/10; A61K 47/44; A61K 47/02; A61K 47/28; A61K 9/0019
USPC 514/301

(57) ABSTRACT

A composition comprises a kinase inhibitory compound, e.g., N-(4-{4-amino-7-[1-(2-hydroxyethyl)-1H-pyrazol-4-yl]thieno[3,2-c]pyridin-3-yl}phenyl)-N′-(3-fluorophenyl)urea, in a mixture comprising (a) a pharmaceutically acceptable water-soluble polymeric carrier and (b) a pharmaceutically acceptable surfactant. The composition is suitable for dilution with an IV solution for administration to a subject in need thereof for treatment of a cancer.

### CROSS-REFERENCE TO RELATED APPLICATIONS

This application claims priority to U.S. Provisional Application Ser. No. 61/570,679, filed Dec. 14, 2011, which claims priority to PCT/US2012/069641, filed Dec. 14, 2012, which is incorporated by reference in its entirety.

### FIELD OF THE INVENTION

The present invention relates to solid dispersions comprising compounds that inhibit protein kinases, to pharmaceutical dosage forms comprising such dispersions, to processes for preparing such dispersions and dosage forms and to methods of use thereof for treating diseases.

### BACKGROUND OF THE INVENTION

Mitosis is a process by which a complete copy of a duplicated genome is segregated by the microtuble spindle apparatus into two daughter cells. Aurora-kinases, key mitotic regulators required for genome stability, have been found to be overexpressed in human tumors. There is therefore an existing need in the therapeutic arts for compounds which inhibit Aurora-kinases, compositions comprising the inhibitors and methods of treating diseases during which Aurora-kinases are unregulated or overexpressed.

The reversible phosphorylation of proteins is one of the primary biochemical mechanisms mediating eukaryotic cell signaling. This reaction is catalyzed by protein kinases that transfer the g-phosphate group of ATP to hydroxyl groups on target proteins. 518 such enzymes exist in the human genome of which ˜90 selectively catalyze the phosphorylation of tyrosine hydroxyl groups Cytosolic tyrosine kinases reside intracellularly whereas receptor tyrosine kinases (RTKs) possess both extracellular and intracellular domains and function as membrane spanning cell surface receptors. As such, RTKs mediate the cellular responses to environmental signals and facilitate a broad range of cellular processes including proliferation, migration and survival.

RTK signaling pathways are normally highly regulated, yet their over-activation has been shown to promote the growth, survival and metastasis of cancer cells. Dysregulated RTK signaling occurs through gene over-expression or mutation and has been correlated with the progression of various human cancers.

The VEGF receptor (VEGFR) family consists of three RTKs, KDR (kinase insert domain-containing receptor; VEGFR2), FLT1 (Fms-like tyrosine kinase; VEGFR1), and FLT4 (VEGFR3). These receptors mediate the biological function of the vascular endothelial growth factors (VEGF-A, -B, -C, -D, -E and placenta growth factor (PlGF)), a family of homodimeric glycoproteins that bind the VEGF receptors with varying affinities.

KDR is the major mediator of the mitogenic, angiogenic and permeability-enhancing effects of VEGF-A, hereafter referred to as VEGF. Many different cell types are able to produce VEGF, yet its biological activity is limited predominately to the vasculature by way of the endothelial cell-selective expression of KDR. Not surprisingly, the VEGF/KDR axis is a primary mediator of angiogenesis, the means by which new blood vessels are formed from preexisting vessels.

FLT1 binds VEGF, VEGF-B and placental growth factor. FLT1 is expressed on the surface of smooth muscle cells, monocytes and hematopoietic stems cells in addition to endothelial cells. Activation of FLT1 signaling results in the mobilization of marrow-derived endothelial progenitor cells that are recruited to tumors where they contribute to new blood vessel formation.

FLT4 mediates the signaling of VEGF-C and VEGF-D, which mediate formation of tumor-associated lymphatic vessels (lymphangiogenesis). Lymphatic vessels are one of the routes by which cancer cells disseminate from solid tumors during metastasis.

The PDGF receptor (PDGFR) family consists of five RTK's, PDGFR-a and -b, CSF1R, KIT, and FLT3.

CSF-1R is encoded by the cellular homolog of the retroviral oncogene v-fms and is a major regulator of macrophage development. Macrophages are frequent components of tumor stroma and have been shown to modify the extracellular matrix in a manner beneficial to tumor growth and metastasis.

KIT is expressed by hematopoietic progenitor cells, mast cells, germ cells and by pacemaker cells in the gut (interstitial cells of Cajal). It contributes to tumor progression by two general mechanisms namely autocrine stimulation by its ligand, stem cell factor (SCF), and through mutations that result in ligand-independent kinase activity.

FLT3 is normally expressed on hematopoietic stem cells where its interaction with FLT3 ligand (FL) stimulates stem cell survival, proliferation and differentiation. In addition to being over-expressed in various leukemia cells, FLT3 is frequently mutated in hematological malignancies with approximately one-third of patients with acute myeloid leukemia (AML) harboring activating mutations.

The identification of effective small compounds which specifically inhibit signal transduction and cellular proliferation by modulating the activity of tyrosine kinases to regulate and modulate abnormal or inappropriate cell proliferation, differentiation, or metabolism is therefore desirable. In particular, the identification of methods and compounds that specifically inhibit the function of a tyrosine kinase which is essential for angiogenic processes or the formation of vascular hyperpermeability leading to edema, ascites, effusions, exudates, and macromolecular extravasation and matrix deposition as well as associated disorders would be beneficial.

Compounds that inhibit protein kinases such as Aurora-kinases and the VEGFR and PDGFR families of kinases have been identified. These compounds, and methods to make them, are disclosed in U.S. Patent Publication No. 2007-0155776 A1 (hereinafter the '776 publication) and U.S. Patent Publication No. 2010-0144783 A1 (hereinafter “the '783 publication”), incorporated by reference herein in their entirety.

The very low aqueous solubility of compounds, for example, of the '783 publication raises challenges for the formulator due to the need to solubilize the compounds for administration to patients, particularly for producing a formulation for use in intravenous administration. The formulation must enhance the solubility of a sparingly water-soluble compound in water to such an extent that a pharmaceutically acceptable amount of the kinase inhibitor can be administrated, i.e., suitably high concentrations of drug, and that the kinase inhibitor is stable in the formulation, i.e., minimizing precipitation of the kinase inhibitor.

To enhance the clinical utility of an inhibitor of protein kinases, for example as a chemotherapeutic in cancer patients, such an IV form would be highly desirable. Such a dosage form, and a regimen for the IV administration thereof, would represent an important advance in treatment of many types of cancer, and would more readily enable combination therapies with other chemotherapeutics.

### SUMMARY OF THE INVENTION

There is now provided a composition comprising N-(4-{4-amino-7-[1-(2-hydroxyethyl)-1H-pyrazol-4-yl]thieno[3,2-c]pyridin-3-yl}phenyl)-N′-(3-fluorophenyl)urea or a salt thereof, polyethylene glycol; polyoxyethylated castor oil; and ethanol, wherein the polyethylene glycol and the polyoxyethylated castor oil are present in a 1:1 ratio by weight.

There is further provided a composition comprising (a) N-(4-{4-amino-7-[1-(2-hydroxyethyl)-1H-pyrazol-4-yl]thieno[3,2-c]pyridin-3-yl}phenyl)-N′-(3-fluorophenyl)urea or a salt thereof; (b) polyethylene glycol (c) polyoxyethylated castor oil; (d) ethanol; and (e) a pharmaceutically acceptable IV solution selected from the group consisting of a saline solution and a dextrose solution; wherein the polyethylene glycol and the polyoxyethylated castor oil are present in a 1:1 ratio by weight.

There is still further provided a method for treating cancer comprising administering to a subject having the disease a therapeutically effective amount of the a composition as described above.

Additional embodiments of the invention, including more particular aspects of those provided above, will be found in, or will be evident from, the detailed description that follows.

### DETAILED DESCRIPTION

A composition in accordance with the present disclosure comprises N-(4-{4-amino-7-[1-(2-hydroxyethyl)-1H-pyrazol-4-yl]thieno[3,2-c]pyridin-3-yl}phenyl)-N′-(3-fluorophenyl)urea or a pharmaceutically acceptable salt thereof in a concentrated mixture comprising water-miscible organic solvents and/or surfactants (“pre-concentrate”). The composition is suitable for dilution in an aqueous solution prior to delivery by intravenous administration.

N-(4-{4-amino-7-[1-(2-hydroxyethyl)-1H-pyrazol-4-yl]thieno[3,2-c]pyridin-3-yl}phenyl)-N′-(3-fluorophenyl)urea, including salts thereof, typically have very low solubility in water, for example less than about 100 μg/ml, in most cases less than about 30 μg/ml. The present invention can be especially advantageous for drugs that are essentially insoluble in water, i.e., having a solubility of less than about 10 μg/ml. It will be recognized that aqueous solubility of many compounds is pH-dependent; in the case of such compounds the solubility of interest herein is at a physiologically relevant pH, for example a pH of about 1 to about 8. Thus, in various embodiments, the drug has a solubility in water, at least at one point in a pH range from about 1 to about 8, of less than about 100 μg/ml, for example less than about 30 μg/ml, or less than about 10 μg/ml. Illustratively, N-(4-{4-amino-7-[1-(2-hydroxyethyl)-1H-pyrazol-4-yl]thieno[3,2-c]pyridin-3-yl}phenyl)-N′-(3-fluorophenyl)urea has a solubility in water of less than 30 ng/ml at pH 7.4.

The active ingredient N-(4-{4-amino-7-[1-(2-hydroxyethyl)-1H-pyrazol-4-yl]thieno[3,2-c]pyridin-3-yl}phenyl)-N′-(3-fluorophenyl)urea for use in this invention can be in salt form or the non-salt free base. In one embodiment, the composition comprises the non-salt free base of N-(4-{4-amino-7-[1-(2-hydroxyethyl)-1H-pyrazol-4-yl]thieno[3,2-c]pyridin-3-yl}phenyl)-N′-(3-fluorophenyl)urea.

N-(4-{4-amino-7-[1-(2-hydroxyethyl)-1H-pyrazol-4-yl]thieno[3,2-c]pyridin-3-yl}phenyl)-N′-(3-fluorophenyl)urea is prepared, illustratively, as described in Example 1 of above-cited '783 publication.

N-(4-{4-amino-7-[1-(2-hydroxyethyl)-1H-pyrazol-4-yl]thieno[3,2-c]pyridin-3-yl}phenyl)-N′-(3-fluorophenyl)urea is present in the composition of the invention in an amount that can be therapeutically effective when the composition is administered to a subject in need thereof according to an appropriate regimen. Dosage amounts are expressed herein as parent-compound-equivalent (free base equivalent) amounts unless the context requires otherwise. Typically, a unit dose (the amount administered at a single time), which can be administered at an appropriate frequency, e.g., twice daily to once monthly, is about 10 to about 1,000 mg, depending on the compound in question. Where frequency of administration is once daily (q.d.), unit dose and daily dose are the same. Illustratively, the unit dose is typically about 25 to about 1,000 mg, more typically about 50 to about 500 mg, for example about 50, about 100, about 150, about 200, about 250, or about 300 mg.

The higher the unit dose, the more desirable it becomes to prepare a composition having a relatively high concentration of the drug therein. Typically, the concentration of drug in the pre-concentrate is about 4 mg/mL to about 10 mg/mL. In one embodiment of the invention, the concentration of drug in the pre-concentrate is about 6 mg/mL.

A major component of the pre-concentrate of the invention is a 1:1 mixture of polyethylene glycol and polyoxyethylated castor oil by weight. The mixture of water-miscible organic solvents and/or surfactants serves to solubilize the N-(4-{4-amino-7-[1-(2-hydroxyethyl)-1H-pyrazol-4-yl]thieno[3,2-c]pyridin-3-yl}phenyl)-N′-(3-fluorophenyl)urea.

Examples of polyethylene glycol useful herein include polyethylene glycol 300 and polyethylene glycol 400. In one embodiment of the invention, the polyethylene glycol is polyethylene glycol 300.

Examples of polyoxyethylated castor oil useful herein include polyoxyl 35 castor oil (Cremophor EL) and polyoxyl 40 hydrogenated castor oil (Cremophor RH 40). In one embodiment, the polyoxyethylated castor oil is polyoxyl 35 castor oil (Cremophor EL).

The 1:1 mixture of polyethylene glycol and polyoxyethylated castor oil typically constitute in total about 85% to about 95% by weight of the composition that is a preconcentrate. In one embodiment of the invention, polyethylene glycol and polyoxyethylated castor oil are each present in the composition in a range of about 42.5% w/w to about 47.5% w/w. In another embodiment, polyethylene glycol and polyoxyethylated castor oil are each present in the composition in 45% w/w.

The composition further comprises additional water-soluble organic solvents. Water-miscible solvents useful herein include ethanol. Ethanol typically constitutes in total about 5% to about 15%, for example about 10% by weight, of the preconcentrate composition.

In one embodiment, the composition that is a pre-concentrate comprises a mixture of polyethylene glycol 300, polyoxyl 35 castor oil, and ethanol in a ratio of 45:45:10% w/w, and the free base of N-(4-{4-amino-7-[1-(2-hydroxyethyl)-1H-pyrazol-4-yl]thieno[3,2-c]pyridin-3-yl}phenyl)-N′-(3-fluorophenyl)urea, wherein the N-(4-{4-amino-7-[1-(2-hydroxyethyl)-1H-pyrazol-4-yl]thieno[3,2-c]pyridin-3-yl}phenyl)-N′-(3-fluorophenyl)urea is present in a concentration of about 6 mg/mL.

In a second aspect, the invention further comprises a composition suitable as an intravenous formulation comprising any of the concentrated compositions previously described comprising N-(4-{4-amino-7-[1-(2-hydroxyethyl)-1H-pyrazol-4-yl]thieno[3,2-c]pyridin-3-yl}phenyl)-N′-(3-fluorophenyl)urea solubilized in a mixture of water-miscible organic solvents and/or surfactants diluted with a pharmaceutically acceptable, aqueous IV solution.

In one embodiment, a composition comprising N-(4-{4-amino-7-[1-(2-hydroxyethyl)-1H-pyrazol-4-yl]thieno[3,2-c]pyridin-3-yl}phenyl)-N′-(3-fluorophenyl)urea or a salt thereof, polyethylene glycol, polyoxyethylated castor oil, and ethanol, wherein the polyethylene glycol and the polyoxyethylated castor oil are present in a 1:1 ratio by weight, is diluted with an aqueous solution comprising 0.45% NaCl. In another embodiment, the preconcentrate is diluted with an aqueous solution comprising 0.9% NaCl. In yet another embodiment, the pre-concentrate is diluted with an aqueous solution comprising 5% dextrose. In one embodiment, the dilution is a 5-20 fold dilution. In another embodiment, the dilution is a 15-16 fold dilution.

Yet another embodiment of the invention relates to a pharmaceutical composition suitable for intravenous administration comprising 200 mg/500 mL of the free base of N-(4-{4-amino-7-[1-(2-hydroxyethyl)-1H-pyrazol-4-yl]thieno[3,2-c]pyridin-3-yl}phenyl)-N-(3-fluorophenyl)urea, in a mixture of polyethylene glycol 300, polyoxyl 35 castor oil, ethanol, and about a pharmaceutically acceptable, aqueous IV solution, wherein the polyethylene glycol 300 and the polyoxyl 35 castor oil are in a 1:1 ratio by weight. In one embodiment, the IV solution contains 0.45% NaCl. In another embodiment, the IV solution contains 0.9% NaCl. In yet another embodiment, the IV solution contains 5% dextrose. In yet another embodiment, the pharmaceutical composition comprises 50 mg/500 mL, 100 mg/500 mL, or 150 mg/500 mL of the free base of N-(4-{4-amino-7-[1-(2-hydroxyethyl)-1H-pyrazol-4-yl]thieno[3,2-c]pyridin-3-yl}phenyl)-N′-(3-fluorophenyl)urea. In yet another embodiment, pharmaceutical composition comprises 100 mg/1000 mL, 150 mg/1000 mL, 200 mg/1000 mL, or 250 mg/1000 mL of the free base of N-(4-{4-amino-7-[1-(2-hydroxyethyl)-1H-pyrazol-4-yl]thieno[3,2-c]pyridin-3-yl}phenyl)-N-(3-fluorophenyl)urea.

The pharmaceutical compositions of the present invention suitable for intravenous administration are stable, i.e., delayed precipitation of the kinase inhibitor N-(4-{4-amino-7-[1-(2-hydroxyethyl)-1H-pyrazol-4-yl]thieno[3,2-c]pyridin-3-yl}phenyl)-N-(3-fluorophenyl)urea after dilution in the IV solution. In particular, the pharmaceutical compositions have less than 25 particles/mL of particles >10 μm in diameter and less than 3 particles/mL of particles >25 μm in diameter at 24 hours after dilution.

The subject can be human or non-human (e.g., a farm, zoo, work or companion animal, or a laboratory animal used as a model) but in an important embodiment the subject is a human patient in need of the drug, for example to treat cancer. A human subject can be male or female and of any age, but is typically an adult.

The composition is normally administered in an amount providing a therapeutically effective daily dose of the drug. The term “daily dose” herein means the amount of drug administered per day, regardless of the frequency of administration. For example, if the subject receives a unit dose of 150 mg twice daily, the daily dose is 300 mg. Use of the term “daily dose” will be understood not to imply that the specified dosage amount is necessarily administered once daily. However, in a particular embodiment the dosing frequency is once daily (q.d.), and the daily dose and unit dose are in this embodiment the same thing.

What constitutes a therapeutically effective dose depends on the particular compound, the subject (including species and body weight of the subject), the disease (e.g., the particular type of cancer) to be treated, the stage and/or severity of the disease, the individual subject's tolerance of the compound, whether the compound is administered in monotherapy or in combination with one or more other drugs, e.g., other chemotherapeutics for treatment of cancer, and other factors. Thus the daily dose can vary within wide margins, for example from about 10 to about 1,000 mg. Greater or lesser daily doses can be appropriate in specific situations. It will be understood that recitation herein of a “therapeutically effective” dose herein does not necessarily require that the drug be therapeutically effective if only a single such dose is administered; typically therapeutic efficacy depends on the composition being administered repeatedly according to a regimen involving appropriate frequency and duration of administration. It is strongly preferred that, while the daily dose selected is sufficient to provide benefit in terms of treating the cancer, it should not be sufficient to provoke an adverse side-effect to an unacceptable or intolerable degree. A suitable therapeutically effective dose can be selected by the physician of ordinary skill without undue experimentation based on the disclosure herein and on art cited herein, taking into account factors such as those mentioned above. The physician may, for example, start a cancer patient on a course of therapy with a relatively low daily dose and titrate the dose upwards over a period of days or weeks, to reduce risk of adverse side-effects.

Illustratively, suitable doses of N-(4-{4-amino-7-[1-(2-hydroxyethyl)-1H-pyrazol-4-yl]thieno[3,2-c]pyridin-3-yl}phenyl)-N′-(3-fluorophenyl)urea are generally about 10 to about 1,000 mg/day, more typically about 50 to about 500 mg/day or about 200 to about 400 mg/day, for example about 50, about 100, about 150, about 200, about 250, about 300, about 350, about 400, about 450 or about 500 mg/day, administered at an average dosage interval of 3 to 10 days, or about 4 to 8 days, or about 7 days.

Compositions of the invention are suitable for use in monotherapy or in combination therapy, for example with other chemotherapeutics or with ionizing radiation.

A composition of the invention, for example such a composition comprising N-(4-{4-amino-7-[1-(2-hydroxyethyl)-1H-pyrazol-4-yl]thieno[3,2-c]pyridin-3-yl}phenyl)-N-(3-fluorophenyl)urea, can be administered in combination therapy with one or more therapeutic agents that include, but are not limited to, alkylating agents, angiogenesis inhibitors, antibodies, antimetabolites, antimitotics, antiproliferatives, antivirals, aurora kinase inhibitors, other apoptosis promoters (for example, Bcl-xL, Bcl-w and Bfl-1 inhibitors), activators of a death receptor pathway, Bcr-Abl kinase inhibitors, BiTE (bi-specific T-cell engager) antibodies, antibody-drug conjugates, biological response modifiers, cyclin-dependent kinase (CDK) inhibitors, cell cycle inhibitors, cyclooxygenase-2 (COX-2) inhibitors, dual variable domain binding proteins (DVDs), human epidermal growth factor receptor 2 (ErbB2 or HER/2neu) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAPs), intercalating antibiotics, kinase inhibitors, kinesin inhibitors, JAK2 inhibitors, mammalian target of rapamycin (mTOR) inhibitors, microRNAs, mitogen-activated extracellular signal-regulated kinase (MEK) inhibitors, multivalent binding proteins, non-steroidal anti-inflammatory drugs (NSAIDs), poly-ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids, deltoids, plant alkaloids, small inhibitory ribonucleic acids (siRNAs), topoisomerase inhibitors, ubiquitin ligase inhibitors, and the like.

BiTE antibodies are bi-specific antibodies that direct T-cells to attack cancer cells by simultaneously binding the two cells. The T-cell then attacks the target cancer cell. Examples of BiTE antibodies include, but are not limited to, adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (Sutton et al. (1997) J. Immunol. 158:5783-5790).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand. For example, siRNAs targeting Mcl-1 have been shown to enhance the activity of ABT-263 or ABT-737 in various tumor cell lines (Tse et al. (2008) Cancer Res. 68:3421-3428 and references therein).

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy-chain DVD polypeptides and two light-chain DVD polypeptides are referred to as DVD Ig's. Each half of a DVD Ig comprises a heavy-chain DVD polypeptide, a light-chain DVD polypeptide, and two antigen binding sites. Each binding site comprises a heavy-chain variable domain and a light-chain variable domain with a total of 6 CDRs involved in antigen binding per antigen binding site.

Alkylating agents include altretamine, AMD-473, AP-5280, apaziquone, bendamustine, brostallicin, busulfan, carboquone, carmustine (BCNU), chlorambucil, Cloretazine™ (laromustine, VNP 40101M), cyclophosphamide, dacarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, treosulfan, trofosfamide and the like.

Angiogenesis inhibitors include epidermal growth factor receptor (EGFR) inhibitors, endothelial-specific receptor tyrosine kinase (Tie-2) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include Alimta™ (pemetrexed disodium, LY231514, MTA), 5-azacitidine, Xeloda™ (capecitabine), carmofur, Leustat™ (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethenylcytidine, fludarabine, 5-fluorouracil (5-FU) alone or in combination with leucovorin, Gemzar™ (gemcitabine), hydroxyurea, Alkeran™ (melphalan), mercaptopurine, 6-mercaptopurine riboside, methotrexate, mycophenolic acid, nelarabine, nolatrexed, ocfosfate, pelitrexol, pentostatin, raltitrexed, ribavirin, S-1, triapine, trimetrexate, TS-1, tiazofurin, tegafur, vidarabine, UFT and the like.

Antivirals include ritonavir, hydroxychloroquine and the like.

Aurora kinase inhibitors include AZD-1152, MLN-8054, VX-680, aurora A-specific kinase inhibitors, aurora B-specific kinase inhibitors, pan-aurora kinase inhibitors and the like.

Bcl-2 family protein inhibitors other than ABT-263 or compounds of Formula I herein include AT-101 ((−)gossypol), Genasense™ Bcl-2-targeting antisense oligonucleotide (G3139 or oblimersen), IPI-194, IPI-565, N-(4-(4-((4′-chloro(1,1′-biphenyl)-2-yl)methyl) piperazin-1-yl)benzoyl)-4-(((1R)-3-(dimethylamino)-1-((phenylsulfanyl)methyl)propyl)amino)-3-nitrobenzenesulfonamide) (ABT-737), GX-070 (obatoclax) and the like.

Bcr-Abl kinase inhibitors include dasatinib (BMS-354825), Gleevec™ (imatinib) and the like.

CDK inhibitors include AZD-5438, BMI-1040, BMS-387032, CVT-2584, flavopyridol, GPC-286199, MCS-5A, PD0332991, PHA-690509, seliciclib (CYC-202 or R-roscovitine), ZK-304709 and the like.

COX-2 inhibitors include ABT-963, Arcoxia™ (etoricoxib), Bextra™ (valdecoxib), BMS-347070, Celebrex™ (celecoxib), COX-189 (lumiracoxib), CT-3, Deramaxx™ (deracoxib), JTE-522, 4-methyl-2-(3,4-dimethylphenyl)-1-(4-sulfamoylphenyl)-1H-pyrrole, MK-663 (etoricoxib), NS-398, parecoxib, RS-57067, SC-58125, SD-8381, SVT-2016, S-2474, T-614, Vioxx™ (rofecoxib) and the like.

EGFR inhibitors include ABX-EGF, anti-EGFR immunoliposomes, EGF-vaccine, EMD-7200, Erbitux™ (cetuximab), HR3, IgA antibodies, Iressa™ (gefitinib), Tarceva™ (erlotinib or OSI-774), TP-38, EGFR fusion protein, Tykerb™ (lapatinib) and the like.

ErbB2 receptor inhibitors include CP-724714, CI-1033 (canertinib), Herceptin™ (trastuzumab), Tykerb™ (lapatinib), Omnitarg™ (2C4, petuzumab), TAK-165, GW-572016 (ionafamib), GW-282974, EKB-569, PI-166, dHER2 (HER2 vaccine), APC-8024 (HER2 vaccine), anti-HER/2neu bispecific antibody, B7.her2IgG3, AS HER2 trifunctional bispecific antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17AAG, CNF-101, CNF-1010, CNF-2024, 17-DMAG, geldanamycin, IPI-504, KOS-953, Mycograb™ (human recombinant antibody to HSP-90), nab-17AAG, NCS-683664, PU24FCl, PU-3, radicicol, SNX-2112, STA-9090, VER-49009 and the like.

Inhibitors of apoptosis proteins include HGS-1029, GDC-0145, GDC-0152, LCL-161, LBW-242 and the like.

Antibody-drug conjugates include anti-CD22-MC-MMAF, anti-CD22-MC-MMAE, anti-CD22-MCC-DM1, CR-011-veMMAE, PSMA-ADC, MEDI-547, SGN-19A, SGN-35, SGN-75 and the like.

Activators of death receptor pathway include TRAIL and antibodies or other agents that target TRAIL or death receptors (e.g., DR4 and DR5) such as apomab, conatumumab, ETR2-ST01, GDC0145 (lexatumumab), HGS-1029, LBY-135, PRO-1762, trastuzumab and the like.

Kinesin inhibitors include Eg5 inhibitors such as AZD-4877 and ARRY-520, CENPE inhibitors such as GSK-923295A, and the like.

JAK2 inhibitors include CEP-701 (lesaurtinib), XL019, INCB-018424 and the like.

MEK inhibitors include ARRY-142886, ARRY-438162, PD-325901, PD-98059 and the like.

mTOR inhibitors include AP-23573, CCI-779, everolimus, RAD-001, rapamycin, temsirolimus, ATP-competitive TORC1/TORC2 inhibitors, including PI-103, PP242, PP30 and Torin 1, and the like.

Non-steroidal anti-inflammatory drugs include Amigesic™ (salsalate), Dolobid™ (diflunisal), Motrin™ (ibuprofen), Orudis™ (ketoprofen), Relafen™ (nabumetone), Feldene™ (piroxicam), ibuprofen cream, Aleve™ and Naprosyn™ (naproxen), Voltaren™ (diclofenac), Indocin™ (indomethacin), Clinoril™ (sulindac), Tolectin™ (tolmetin), Lodine™ (etodolac), Toradol™ (ketorolac), Daypro™ (oxaprozin) and the like.

PDGFR inhibitors include CP-673451, CP-868596 and the like.

Platinum chemotherapeutics include cisplatin, Eloxatin™ (oxaliplatin), eptaplatin, lobaplatin, nedaplatin, Paraplatin™ (carboplatin), picoplatin, satraplatin and the like.

Polo-like kinase inhibitors include BI-2536 and the like.

Phosphoinositide-3 kinase inhibitors include wortmannin, LY-294002, XL-147, CAL-120, ONC-21, AEZS-127, ETP-45658, PX-866, GDC-0941, BGT226, BEZ235, XL765 and the like.

Thrombospondin analogs include ABT-510, ABT-567, ABT-898, TSP-1 and the like.

VEGFR inhibitors include Avastin™ (bevacizumab), ABT-869, AEE-788, Angiozyme™ (a ribozyme that inhibits angiogenesis (Ribozyme Pharmaceuticals (Boulder, Colo.) and Chiron (Emeryville, Calif.)), axitinib (AG-13736), AZD-2171, CP-547632, IM-862, Macugen™ (pegaptanib), Nexavar™ (sorafenib, BAY43-9006), pazopanib (GW-786034), vatalanib (PTK-787 or ZK-222584), Sutent™ (sunitinib or SU-11248), VEGF trap, Zactima™ (vandetanib or ZD-6474) and the like.

Antibiotics include intercalating antibiotics such as aclarubicin, actinomycin D, amrubicin, annamycin, Adriamycin™ (doxorubicin), Blenoxane™ (bleomycin), daunorubicin, Caelyx™ and Myocet™ (liposomal doxorubicin), elsamitrucin, epirubicin, glarubicin, idarubicin, mitomycin C, nemorubicin, neocarzinostatin, peplomycin, pirarubicin, rebeccamycin, stimalamer, streptozocin, Valstar™ (valrubicin), zinostatin and the like.

Topoisomerase inhibitors include aclarubicin, 9-aminocamptothecin, amonafide, amsacrine, becatecarin, belotecan, BN-80915, Camptosar™ (irinotecan hydrochloride), camptothecin, Cardioxane™ (dexrazoxane), diflomotecan, edotecarin, Ellence™ and Pharmorubicin™ (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include Avastin™ (bevacizumab), CD40-specific antibodies, chTNT-1/B, denosumab, Erbitux™ (cetuximab), Humax-CD4™ (zanolimumab), IGF1R-specific antibodies, lintuzumab, Panorex™ (edrecolomab), Rencarex™ (WX G250), Rituxan™ (rituximab), ticilimumab, trastuzumab, CD20 antibodies types I and II and the like.

Hormonal therapies include Arimidex™ (anastrozole), Aromasin™ (exemestane), arzoxifene, Casodex™ (bicalutamide), Cetrotide™ (cetrorelix), degarelix, deslorelin, Desopan™ (trilostane), dexamethasone, Drogenil™ (flutamide), Evista™ (raloxifene), Afema™ (fadrozole), Fareston™ (toremifene), Faslodex™ (fulvestrant), Femara™ (letrozole), formestane, glucocorticoids, Hectorol™ (doxercalciferol), Renagel™ (sevelamer carbonate), lasofoxifene, leuprolide acetate, Megace™ (megestrol), Mifeprex™ (mifepristone), Nilandron™ (nilutamide), tamoxifen including Nolvadex™ (tamoxifen citrate), Plenaxis™ (abarelix), prednisone, Propecia™ (finasteride), rilostane, Suprefact™ (buserelin), luteinizing hormone releasing hormone (LHRH) including Trelstar™ (triptorelin), histrelin including Vantas™ (histrelin implant), Modrastane™ (trilostane), Zoladex™ (goserelin) and the like.

Deltoids and retinoids include seocalcitol (EB1089 or CB1093), lexacalcitol (KH1060), fenretinide, Panretin™ (alitretinoin), tretinoin including Atragen™ (liposomal tretinoin), Targretin™ (bexarotene), LGD-1550 and the like.

PARP inhibitors include ABT-888, olaparib, KU-59436, AZD-2281, AG-014699, BSI-201, BGP-15, INO-1001, ONO-2231 and the like.

Plant alkaloids include vincristine, vinblastine, vindesine, vinorelbine and the like.

Proteasome inhibitors include Velcade™ (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, Actimmune™ (interferon gamma-1b), interferon gamma-n1, combinations thereof and the like. Other agents include Alfaferone (IFN-α), BAM-002 (oxidized glutathione), Beromun™ (tasonermin), Bexxar™ (tositumomab), Campath™ (alemtuzumab), CTLA4 (cytotoxic lymphocyte antigen 4), dacarbazine, denileukin, epratuzumab, Granocyte™ (lenograstim), lentinan, leukocyte alpha interferon, imiquimod, MDX-010 (anti-CTLA-4), melanoma vaccine, mitumomab, molgramostim, Mylotarg™ (gemtuzumab ozogamicin), Neupogen™ (filgrastim), OncoVAC-CL, Ovarex™ (oregovomab), pemtumomab (Y-muHMFG1), Provenge™ (sipuleucel-T), sargaramostim, sizofiran, teceleukin, Theracys™ (BCG or Bacillus Calmette-Guerin), ubenimex, Virulizin™ (immunotherapeutic, Lorus Pharmaceuticals), Z-100 (Specific Substance of Maruyama or SSM), WF-10 (tetrachlorodecaoxide or TCDO), Proleukin™ (aldesleukin), Zadaxin™ (thymalfasin), Zenapax™ (daclizumab), Zevalin™ (90Y-ibritumomab tiuxetan) and the like.

Biological response modifiers are agents that modify defense mechanisms of living organisms or biological responses, such as survival, growth or differentiation of tissue cells to direct them to have anti-tumor activity, and include krestin, lentinan, sizofiran, picibanil, PF-3512676 (CpG-8954), ubenimex and the like.

Pyrimidine analogs include cytarabine (cytosine arabinoside, ara C or arabinoside C), doxifluridine, Fludara™ (fludarabine), 5-FU (5-fluorouracil), floxuridine, Gemzar™ (gemcitabine), Tomudex™ (raltitrexed), triacetyluridine, Troxatyl™ (troxacitabine) and the like.

Purine analogs include Lanvis™ (thioguanine), Purinethol™ (mercaptopurine) and the like.

Antimitotic agents include batabulin, epothilone D (KOS-862), N-(2-((4-hydroxyphenyl)amino)pyridin-3-yl)-4-methoxybenzenesulfonamide, ixabepilone (BMS-247550), paclitaxel, Taxotere™ (docetaxel), larotaxel (PNU-100940, RPR-109881 or XRP-9881), patupilone, vinflunine, ZK-EPO (synthetic epothilone) and the like.

Ubiquitin ligase inhibitors include MDM2 inhibitors such as nutlins, NEDD8 inhibitors such as MLN4924, and the like.

Compositions of this invention can also be used as radiosensitizers that enhance the efficacy of radiotherapy. Examples of radiotherapy include, but are not limited to, external beam radiotherapy (XBRT), teletherapy, brachytherapy, sealed-source radiotherapy, unsealed-source radiotherapy and the like.

Additionally or alternatively, a composition of the present invention can be administered in combination therapy with one or more antitumor or chemotherapeutic agents selected from Abraxane™ (ABI-007), ABT-100 (farnesyl transferase inhibitor), Advexin™ (AdSCMV-p53 vaccine or contusugene ladenovec), Altocor™ or Mevacor™ (lovastatin), Ampligen™ (poly(I)-poly(C12U), a synthetic RNA), Aptosyn™ (exisulind), Aredia™ (pamidronic acid), arglabin, L-asparaginase, atamestane (1-methyl-3,17-dione-androsta-1,4-diene), Avage™ (tazarotene), AVE-8062 (combretastatin derivative), BEC2 (mitumomab), cachectin or cachexin (tumor necrosis factor), Canvaxin™ (melanoma vaccine), CeaVac™ (cancer vaccine), Celeuk™ (celmoleukin), histamine including Ceplene™ (histamine dihydrochloride), Cervarix™ (ASO4 adjuvant-adsorbed human papilloma virus (HPV) vaccine), CHOP (Cytoxan™ (cyclophosphamide)+Adriamycin™ (doxorubicin)+Oncovin™ (vincristine)+prednisone), combretastatin A4P, Cypat™ (cyproterone), DAB(389)EGF (catalytic and translocation domains of diphtheria toxin fused via a His-Ala linker to human epidermal growth factor), dacarbazine, dactinomycin, Dimericine™ (T4N5 liposome lotion), 5,6-dimethylxanthenone-4-acetic acid (DMXAA), discodermolide, DX-8951f (exatecan mesylate), eniluracil (ethynyluracil), squalamine including Evizon™ (squalamine lactate), enzastaurin, EPO-906 (epothilone B), Gardasil™ (quadrivalent human papilloma virus (Types 6, 11, 16, 18) recombinant vaccine), Gastrimmune™, Genasense™ (oblimersen), GMK (ganglioside conjugate vaccine), GVAX™ (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, Junovan™ and Mepact™ (mifamurtide), lonafarnib, 5,10-methylenetetrahydrofolate, miltefosine (hexadecylphosphocholine), Neovastat™ (AE-941), Neutrexin™ (trimetrexate glucuronate), Nipent™ (pentostatin), Onconase™ (ranpirnase, a ribonuclease enzyme), Oncophage™ (vitespen, melanoma vaccine treatment), OncoVAX™ (IL-2 vaccine), Orathecin™ (rubitecan), Osidem™ (antibody-based cell drug), Ovarex™ MAb (murine monoclonal antibody), paclitaxel albumin-stabilized nanoparticle, paclitaxel, Pandimex™ (aglycone saponins from ginseng comprising 20(S)-protopanaxadiol (aPPD) and 20(S)-protopanaxatriol (aPPT)), panitumumab, Panvac™-VF (investigational cancer vaccine), pegaspargase, peginterferon alfa (PEG interferon A), phenoxodiol, procarbazine, rebimastat, Removab™ (catumaxomab), Revlimid™ (lenalidomide), RSR13 (efaproxiral), Somatuline™ LA (lanreotide), Soriatane™ (acitretin), staurosporine (Streptomyces staurospores), talabostat (PT100), Targretin™ (bexarotene), Taxoprexin™ (docosahexaenoic acid (DHA)+paclitaxel), Telcyta™ (canfosfamide, TLK-286), Temodar™ (temozolomide), tesmilifene, tetrandrine, thalidomide, Theratope™ (STn-KLH vaccine), Thymitaq™ (nolatrexed dihydrochloride), TNFerade™ (adenovector: DNA carrier containing the gene for tumor necrosis factor-α), Tracleer™ or Zavesca™ (bosentan), TransMID-107R™ (KSB-311, diphtheria toxins), tretinoin (retin-A), Trisenox™ (arsenic trioxide), Ukrain™ (derivative of alkaloids from the greater celandine plant), Virulizin™, Vitaxin™ (anti-αvβ3 antibody), Xcytrin™ (motexafin gadolinium), Xinlay™ (atrasentan), Xyotax™ (paclitaxel poliglumex), Yondelis™ (trabectedin), ZD-6126 (N-acetylcolchinol-O-phosphate), Zinecard™ (dexrazoxane), zoledronic acid, zorubicin and the like.

In one embodiment, a composition of the invention, for example such a composition comprising N-(4-{4-amino-7-[1-(2-hydroxyethyl)-1H-pyrazol-4-yl]thieno[3,2-c]pyridin-3-yl}phenyl)-N′-(3-fluorophenyl)urea or a salt thereof, is administered in a therapeutically effective amount to a subject in need thereof to treat cancer.

Examples include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumors, uterine cancer and Wilms' tumor in a mammal,

In a more particular embodiment, a composition of the invention, is administered in a therapeutically effective amount to a subject in need thereof to treat myelodysplastic syndrome, acute myeloid leukemia, colorectal cancer, non-small cell lung cancer, and ovarian cancer.

According to any of these embodiments, the composition is administered in combination therapy with one or more additional therapeutic agents.

### EXAMPLES

The following examples are merely illustrative, and do not limit this disclosure in any way.

### Example 1

### Solubility of N-(4-{4-amino-7-[1-(2-hydroxyethyl)-1H-pyrazol-4-yl]thieno[3,2-c]pyridin-3-yl}phenyl)-N′-(3-fluorophenyl)urea Free Base in Various Media

TABLE 1

| Solvent/Medium | Solubility | |
|---|---|---|
| Water (across physiological pH range) | <30 | ng/mL |
| 10% Cremophor EL in water | 0.106 | mg/mL |
| 10% Polysorbate 80 in water | 0.146 | mg/mL |
| 10% Solutol HS in water | 0.152 | mg/mL |
| Ethanol | 0.130 | mg/mL |
| PEG 400 | 20.9 | mg/mL |
| PEG 300 | 20.0 | mg/mL |
| Cremphor EL | 7.1 | mg/mL |
| Polysorbate 80 | 2.5 | mg/mL |
| Propylene Glycol | 1.2 | mg/mL |
| DMA | 35-50 | mg/mL |

### Example 2

### Visual Observation of the Vehicle at Different Cremophor EL/PEG-300/Ethanol Ratios

TABLE 2

| | Cremophor EL | | |
|---|---|---|---|
| PEG-300 (% V/V) | (% V/V) | Ethanol (% V/V) | Observation |
| 47.5 | 47.5 | 5 | Clear |
| 50 | 45 | 5 | Turbid |
| 55 | 40 | 5 | Turbid |
| 45 | 45 | 10 | Clear |
| 50 | 40 | 10 | Turbid |
| 60 | 30 | 10 | Turbid |
| 42.5 | 42.5 | 15 | Clear |
| 55 | 30 | 15 | Turbid |
| 50 | 30 | 20 | Turbid |
| 40 | 40 | 20 | Clear |
| 50 | 50 | — | Turbid |
| 60 | 40 | — | Turbid |
| 70 | 30 | — | Turbid |

A mixture containing PEG-300 and Cremophor EL results in a turbid vehicle for solubilizing the drug. A mixture containing PEG-300, Cremophor EL, and ethanol results in a clear, single phase solution only when the PEG-300 and Cremophor EL are present in a 1:1 ratio. Otherwise, a mixture containing PEG-300, Cremophor EL, and ethanol also results in a turbid vehicle.

### Example 3

### Solubility of N-(4-{4-amino-7-[1-(2-hydroxyethyl)-1H-pyrazol-4-yl]thieno[3,2-c]pyridin-3-yl}phenyl)-N′-(3-fluorophenyl)urea Free Base in PEG-300/Cremophor EL/Ethanol Vehicles

TABLE 3

| PEG-300/Cremophor | Solubility | |
|---|---|---|
| EL/Ethanol ratio | (mg/mL) at 5° C. | Solubility (mg/mL) at RT |
| 47.5/47.5/5 | 16.4 | 13.0 |
| 45/45/10 | 14.1 | 11.6 |
| 42.5/42.5/15 | 11.9 | 10.0 |

### Example 4

### Preparation of Compositions of N-(4-{4-amino-7-[1-(2-hydroxyethyl)-1H-pyrazol-4-yl]thieno[3,2-c]pyridin-3-yl}phenyl)-N′-(3-fluorophenyl)urea Free Base with Water-Miscible Organic Solvents (“Pre-Concentrate”)

N-(4-{4-amino-7-[1-(2-hydroxyethyl)-1H-pyrazol-4-yl]thieno[3,2-c]pyridin-3-yl}phenyl)-N-(3-fluorophenyl)urea free base (hereinafter “API”) was mixed with organic solvents and/or surfactants the following weight ratios:

Example 4A: 6 mg/mL API in 45% Cremophor EL:45% PEG-300:10% Ethanol
Example 4B: 9 mg/mL API in 45% Cremophor EL:45% PEG-300:10% Ethanol
Example 4C: 10 mg/mL API in 47.5% Cremophor EL:47.5% PEG-300:5% Ethanol
Example 4D (Comparative): 12 mg/mL API in 70% PEG-300:30% Tween 80
Example 4E (Comparative): 10 mg/mL API in 75% PEG-300:25% Tween 80

### Example 5

### Particle Counts of Formulations after Dilution in an IV Solution

The stability of the pharmaceutical formulations suitable for IV administration was determined by measuring the particle counts in solution over time. The pre-concentrate composition, the IV solution, the total API concentration in the diluted composition, and the number of particles as a factor of time in the diluted composition are presented in Tables 4 and 5.

TABLE 4

| | | | Time (hrs) | Number of |
|---|---|---|---|---|
| Preconcentrate | IV solution | API concentration | after dilution | particles/mL |
| 4A | 0.9% NaCl | 100 mg/500 mL | 24 | >=10 μm: 3.00 |
| | | | | >=25 μm: 0.20 |
| 4A | 0.9% NaCl | 160 mg/500 mL | 24 | >=10 μm: 4.40 |
| | | | | >=25 μm: 0.20 |
| 4B | 0.9% NaCl | 200 mg/500 mL | 24 | >=10 μm: 6.5 |
| | | | | >=25 μm: 0.3 |
| 4B | 0.9% NaCl | 100 mg/500 mL | 24 | >=10 μm: 2.8 |
| | | | | >=25 μm: 0 |
| 4B | 0.9% NaCl | 40 mg/500 mL | 24 | >=10 μm: 4.1 |
| | | | | >=25 μm: 0.4 |
| 4B | 5% Dextrose | 200 mg/500 mL | 24 | >=10 μm: 10.2 |
| | | | | >=25 μm: 1 |
| 4B | 5% Dextrose | 100 mg/500 mL | 24 | >=10 μm: 2.7 |
| | | | | >=25 μm: 0.5 |
| 4B | 5% Dextrose | 40 mg/500 mL | 24 | >=10 μm: 2.7 |
| | | | | >=25 μm: 0.7 |
| 4C | 0.9% NaCl | 200 mg/500 mL | 24 | >=10 μm: 3.3 |
| | | | | >=25 μm: 0.8 |
| 4C | 0.9% NaCl | 100 mg/500 mL | 24 | >=10 μm: 6.2 |
| | | | | >=25 μm: 0.3 |
| 4C | 0.9% NaCl | 78 mg/500 mL | 24 | >=10 μm: 0 |
| | | | | >=25 μm: 0 |
| 4C | 0.9% NaCl | 40 mg/500 mL | 24 | >=10 μm: 6 |
| | | | | >=25 μm: 0.7 |
| 4C | 0.9% NaCl | 19 mg/500 mL | 24 | >=10 μm: 0.2 |
| | | | | >=25 μm: 0 |
| 4C | 5% Dextrose | 200 mg/500 mL | 24 | >=10 μm: 3.2 |
| | | | | >=25 μm: 0 |
| 4C | 5% Dextrose | 100 mg/500 mL | 24 | >=10 μm: 3.5 |
| | | | | >=25 μm: 0 |
| 4C | 5% Dextrose | 78 mg/500 mL | 24 | >=10 μm: 2.8 |
| | | | | >=25 μm: 0.4 |
| 4C | 5% Dextrose | 40 mg/500 mL | 24 | >=10 μm: 0.8 |
| | | | | >=25 μm: 0.3 |
| 4C | 5% Dextrose | 19 mg/500 mL | 24 | >=10 μm: 3.2 |
| | | | | >=25 μm: 0.5 |
| 4D | 0.9% NaCl | 160 mg/500 mL | 2 | >=10 μm: 40 |
| 4D | 0.9% NaCl | 160 mg/1000 mL | 4 | >=10 μm: 36 |
| 4D | 5% Dextrose | 160 mg/1000 mL | 4 | >=10 μm: 36 |
| 4E | 0.9% NaCl | 160 mg/1000 mL | 6 | >=10 μm: 23 |
| 4E | 5% Dextrose | 160 mg/1000 mL | 6 | >=10 μm: 40 |

TABLE 5

| Pre- | IV | API | Particle | Cumulative count/mL | | | |
|---|---|---|---|---|---|---|---|
| concen- | solu- | Concen- | size | t = 0 | 8 | 24 | 24 |
| trate | tion | tration | (μm) | hr | hrs | hrs | hrs |
| 4A | 0.9% | 100 mg/ | 10 | 1.93 | 3.73 | 3.00 | 16.33 |
| | NaCl | 500 mL | 25 | 0.20 | 0.53 | 0.20 | 0.47 |
| 4A | 0.9% | 100 mg/ | 10 | 7.07 | 9.13 | 4.40 | 4.73 |
| | NaCl | 500 mL | 25 | 0.27 | 0.27 | 0.20 | 0.13 |

### Example 6

### Particle Counts of Formulations after Dilution in an IV Solution in a Dynamic Experiment

The stability of a pharmaceutical formulation suitable for IV administration was determined by measuring the particles counts in solution over time after pumping the composition through an IV line at 125 mL/hr. The formulation was produced by diluting preconcentrate 4A in 500 mL 0.9% NaCl. The total API concentration in the diluted composition, and the number of particles as a factor of time in the diluted composition are presented in Table 6.

TABLE 6

| | Storage | | Par- | Cumulative Counts/mL | | | |
|---|---|---|---|---|---|---|---|
| API | time | | ticle | after IV pump | | | |
| concen- | before IV | fil- | size | 1 hr IV | 2 hr IV | 3 hr IV | 4 hr IV |
| tration | pump (hr) | ter | (μm) | pump | pump | pump | pump |
| 200 mg/ | 20 | No | 10 | 3.04 | 2.53 | 2.07 | 1.97 |
| 500 mL | | No | 25 | 0.04 | 0.1 | 0.13 | 0.07 |
| 200 mg/ | 20 | Yes | 10 | 2.04 | 0.4 | 0.2 | 0.1 |
| 500 mL | | Yes | 25 | 0.1 | 0.04 | 0.0 | 0.0 |
| 19 mg/ | 21 | No | 10 | 1.84 | 1.14 | 2.27 | 1.10 |
| 500 mL | | No | 25 | 0.20 | 0.04 | 0.14 | 0.07 |
| 19 mg/ | 21 | Yes | 10 | 1.54 | 0.50 | 0.14 | 0.2 |
| 500 mL | | Yes | 25 | 0.04 | 0.04 | 0.0 | 0.0 |

The compositions of the present invention suitable for IV administration are stable after dilution for at least 24 hours.

### Example 7

### Pharmacokinetics of IV Formulation in Humans

IV formulations of the invention were used in an open-label Phase I human study evaluating the safety and pharmacokinetics of N-(4-{4-amino-7-[1-(2-hydroxyethyl)-1H-pyrazol-4-yl]thieno[3,2-c]pyridin-3-yl}phenyl)-N′-(3-fluorophenyl)urea as monotherapy in subjects with advanced solid tumors, i.e., mixture of polyethylene glycol 300, polyoxyl 35 castor oil, and ethanol in a ratio of 45:45:10% w/w, and N-(4-{4-amino-7-[1-(2-hydroxyethyl)-1H-pyrazol-4-yl]thieno[3,2-c]pyridin-3-yl}phenyl)-N′-(3-fluorophenyl)urea, wherein the N-(4-{4-amino-7-[1-(2-hydroxyethyl)-1H-pyrazol-4-yl]thieno[3,2-c]pyridin-3-yl}phenyl)-N′-(3-fluorophenyl)urea is present in a concentration of about 6 mg/mL.

The number of subjects that entered the studies and completed at least a portion of the studies are noted. Subjects entered the study and were assigned to receive one of the following doses: 8 mg, 16 mg, or 32 mg.

Doses were administered on Day 1 and Day 15 of each 28 day cycle with an approximately 2 hour infusion. On Day 1 and Day 15, plasma samples are collected at time 0 (prior to infusion), 1 h 55 min (just before the end of the infusion), and at 0.5, 1, 2, 4, 6, 8, 10, 24 hours post the end of infusion. The plasma concentrations of N-(4-{4-amino-7-[1-(2-hydroxyethyl)-1H-pyrazol-4-yl]thieno[3,2-c]pyridin-3-yl}phenyl)-N′-(3-fluorophenyl)urea were determined, and the values for the pharmacokinetic parameters were calculated and shown in Table 7.

TABLE 7

API Dose (mg)

| | | | |
|---|---|---|---|
| Parameter | 8^{b} | 16 | 32^{c} |

Study Day 1

| N | 4 | | 1 | 2 | |
|---|---|---|---|---|---|
| t_{1/2} (h)^{a} | 10.7 ± 8.3 | (92.2) | 9.8 | 8.9 | (9.2, 8.5) |
| Tₘₐₓ (h) | 1.9 ± 0.0 | (0.0) | 2.5 | 1.9 | (1.9, 1.9) |
| Cₘₐₓ (ng/mL) | 235 ± 82.1 | (35.0) | 366 | 1313 | (1690, 935) |
| AUC_{∞} | 2.96 ± 1.54 | (51.9) | 2.35 | 8.73 | (12.8, 4.70) |
| (μg · h/mL) | | | | | |

Study Day 15

| | | | |
|---|---|---|---|
| N | 4 | 1 | 2 |

| | | | | | |
|---|---|---|---|---|---|
| t_{1/2} (h)^{a} | 14.1 ± 7.4 | (53.5) | 12.6 | 11.3 | (13.8, 9.6) |
| Tₘₐₓ (h) | 1.9 ± 0.0 | (0.0) | 1.9 | 2.2 | (2.5, 1.9) |
| Cₘₐₓ (ng/mL) | 246 ± 107 | (43.3) | 736 | 1366 | (1850, 882) |
| AUC_{∞} | 2.94 ± 1.63 | (55.4) | 3.69 | 8.62 | (11.8, 5.5) |
| (μg · h/mL) | | | | | |

^{a}Harmonic Mean and Pseudo Standard Deviation
^{b}Parameters reported as Mean ± SD (% CV)
^{c}Parameters reported as Mean (individual parameters)

What is claimed is:

1. A composition comprising
(a) N-(4-{4-amino-7-[1-(2-hydroxyethyl)-1H-pyrazol-4-yl]thieno[3,2-c]pyridin-3-yl}phenyl)-N′-(3-fluorophenyl)urea or a salt thereof;
(b) polyethylene glycol;
(c) polyoxyethylated castor oil; and
(d) ethanol;
wherein the polyethylene glycol and the polyoxyethylated castor oil are present in a 1:1 ratio by weight.

2. The composition of claim 1 comprising the free base of N-(4-{4-amino-7-[1-(2-hydroxyethyl)-1H-pyrazol-4-yl]thieno[3,2-c]pyridin-3-yl}phenyl)-N′-(3-fluorophenyl)urea.

3. The composition of claim 1, wherein the N-(4-{4-amino-7-[1-(2-hydroxyethyl)-1H-pyrazol-4-yl]thieno[3,2-c]pyridin-3-yl}phenyl)-N′-(3-fluorophenyl)urea or a salt thereof is present in a concentration of about 4 mg/mL to about 10 mg/mL.

4. The composition of claim 1, wherein the N-(4-{4-amino-7-[1-(2-hydroxyethyl)-1H-pyrazol-4-yl]thieno[3,2-c]pyridin-3-yl}phenyl)-N′-(3-fluorophenyl)urea or a salt thereof is present in a concentration of about 6 mg/mL.

5. The composition of claim 1, wherein the polyethylene glycol and the polyoxyethylated castor oil are each present in a range of about 42.5% w/w to about 47.5% w/w.

6. The composition of claim 5, wherein the polyethylene glycol and the polyoxethylated castor oil are each present in about 45% w/w.

7. The composition of claim 1, wherein the ethanol is present in about 10% by w/w.

8. The composition of claim 1, wherein the polyethylene glycol is polyethylene glycol 300.

9. The composition of claim 1, wherein the polyoxethylated castor oil is polyoxyl 35 castor oil.

10. The composition of claim 1, comprising a mixture of
polyethylene glycol 300, polyoxyl 35 castor oil, and ethanol in a ratio of 45:45:10% w/w, and N-(4-{4-amino-7-[1-(2-hydroxyethyl)-1H-pyrazol-4-yl]thieno[3,2-c]pyridin-3-yl}phenyl)-N′-(3-fluorophenyl)urea, wherein the N-(4-{4-amino-7-[1-(2-hydroxyethyl)-1H-pyrazol-4-yl]thieno[3,2-c]pyridin-3-yl}phenyl)-N′-(3-fluorophenyl)urea is present in a concentration of about 6 mg/mL.

11. The composition of claim 1, wherein the composition is diluted in an aqueous solution comprising 0.45% NaCl.

12. The composition of claim 1, wherein the composition is diluted in an aqueous solution comprising 0.9% NaCl.

13. The composition of claim 1, wherein the composition is diluted in an aqueous solution comprising 5% dextrose.

14. The composition of claim 1, wherein the dilution is a 5-20 fold dilution.

15. The composition of claim 14, wherein the dilution is a 15-16 fold dilution.

16. A pharmaceutical composition comprising (a) N-(4-{4-amino-7-[1-(2-hydroxyethyl)-1H-pyrazol-4-yl]thieno[3,2-c]pyridin-3-yl}phenyl)-N′-(3-fluorophenyl)urea or a salt thereof;
(b) polyethylene glycol;
(c) polyoxyethylated castor oil;
(d) ethanol; and
(e) a pharmaceutically acceptable IV solution selected from the group consisting of a saline solution and a dextrose solution;
wherein the polyethylene glycol and the polyoxyethylated castor oil are present in a 1:1 ratio by weight.

17. The pharmaceutical composition of claim 16, comprising about 200 mg of the free base of N-(4-{4-amino-7-[1-(2-hydroxyethyl)-1H-pyrazol-4-yl]thieno[3,2-c]pyridin-3-yl}phenyl)-N′-(3-fluorophenyl)urea.

18. The pharmaceutical composition of claim 16, wherein the polyethylene glycol is polyethylene glycol 300.

19. The pharmaceutical composition of claim 16, wherein the polyoxethylated castor oil is polyoxyl 35 castor oil.

20. The pharmaceutical composition of claim 16, wherein the IV solution is a 0.45% saline solution.

21. The pharmaceutical composition of claim 16, wherein the IV solution is a 0.9% saline solution.

22. The pharmaceutical composition of claim 16, wherein the IV solution is a 5% dextrose solution.

23. A method of treating cancer comprising administering to a subject having the disease a therapeutically effective amount of the pharmaceutical composition of claim 1.

24. The method of claim 23, wherein the pharmaceutical composition is administered by intravenous administration.
